(19) United States
(12) Patent Application Publication
(10) Pub. No.: US 2005/0261638 A1
(43) Pub. Date: Nov. 24, 2005

(54) Needle unit

(76) Inventor: Alastair D. Wright, Edinburgh (GB)

Correspondence Address: BAKER & DANIELS LLP;111 E. WAYNE STREET, SUITE 800, FORT WAYNE, IN, 46802, US

(21) Appl. No.: 10/528,744

(22) PCT Filed: Sep. 25, 2003
(86) PCT No.: PCT/GB03/04099

§ 371 Date: Mar. 22, 2005

(30) Foreign Application Priority Data
Sep. 25, 2002 (GB) 0222166.1

(51) Int. Cl. A61M 5/32

(52) U.S. Cl. 604/272

(57) ABSTRACT

A syringe comprising a barrel (10), a plunger (11) and a needle unit (12). The needle unit (12) has a needle (30) which is biased inwardly of the barrel (10) but whose inward movement is blocked by a stop element (34). The needle (30) is released from the stop element (34) in response to the plunger (11) reaching the final part of, or the conclusion of, its delivery stroke to allow retraction of the needle (30). The barrel (10) of the syringe is provided with a seal (38) which contacts an outer peripheral surface of the stop element (34).

This invention relates to fluid-handling devices, in particular, hypodermic syringes, and is particularly concerned with needle units adapted to be coupled to syringe barrels from which fluid is to be dispensed via the needle.

In our prior International Patent Application No. PCT/GBO2/01865 (WO-A-02/087669), there is disclosed a syringe comprising a barrel and a needle unit comprising a housing connected or connectable at one end to a barrel , a needle-mounting hub, a biasing element arranged to urge the hub inwardly of the barrel, and a stop element blocking inward movement of the hub into the barrel until the hub is released from the stop element in response to a plunger associated with the barrel reaching the final part of, or the conclusion of, its delivery stroke to allow retraction of the needle-mounting hub into the hollow plunger. Such a needle unit is referred to herein as a “needle unit as defined”.

In the needle unit of International Patent Application No. PCT/GB02/01865 (WO-A-02/087669), the stop element includes an integral seal, in particular a lip seal, to ensure that the liquid delivered from the barrel passes through the needle.

One problem with a needle unit as defined is that the needle retraction mechanism may inadvertently be activated prematurely. For example, during transport of a syringe comprising the needle unit, the plunger may be depressed with sufficient force to activate the retraction mechanism. Alternatively, if the needle unit is stored separately from the barrel of the syringe, then the stop element may be accidentally knocked, releasing the hub and retracting the needle. Once the needle has been retracted, the needle unit cannot be used and is wasted.

The present invention seeks to provide improvements in needle units as defined and syringes comprising such needle units. However, it is to be appreciated that the provision of a hollow plunger is not an essential feature of the invention since, for example, the plunger may comprise a piston mender and a separate rod in which case the needle-mounting hub is retracted into the barrel adjacent to the rod rather than within the plunger as such.

According to one aspect of the present invention there is provided a syringe having a needle unit as defined and in which the barrel or a part for connecting the needle housing to the barrel is provided with a seal which contacts an outer peripheral surface of the stop element.

The seal may be a lip seal which tends to deflect radially inwardly when the fluid is pressurised during the delivery stroke of the plunger.

The seal may be integral with the barrel or said connecting part.

The housing or the connecting part may be readily connectable to the barrel but once connected the arrangement may be such that it cannot be freed from the barrel, at least not without the application of forces substantially in excess of the force required to effect the connection to the barrel.

The housing or the connecting part may be designed to snap engage with the barrel.

According to a second aspect of the present invention there is provided a needle unit in which the stop element is arranged to snap engage within the housing to couple the needle hub to the housing and retain the biasing element in a stored energy condition.

The biasing element is typically a coiled compression spring which is arranged in encircling relation with the needle.

According to a further aspect of the present invention there is provided a needle unit as defined and a sheath for enclosing the needle, the housing including one or more openings through which the sheath and stop element can make contact. The sheath and stop element may make contact so that the sheath can be used to apply axial force to the stop element without significantly stressing the coupling between the stop element and the hub.

This aspect of the invention is particularly advantageous where a sliding type sealing action is provided between the outer periphery of the stop element and an adjacent component. The sliding type sealing action may for example be provided by means of a lip seal on the barrel or an intermediate part for connecting the housing to the barrel.

Alternatively, or in addition, the sheath and stop element may make contact so that the sheath restricts movement. of the stop element, to prevent release of the needle mounting hub from the stop element. Thus retraction of the needle is prevented until the sheath is removed.

In one embodiment, the housing is provided with one or more openings with which a projection or projections on the stop element snap engage and the openings are so arranged that a portion or portions of the sheath can be brought into abutment with said projection(s). In another embodiment, the projection or projections on the stop element engage with an inwardly directed rib or the like on the housing. The projection or projections may have an inclined outer face such that they may ride over the inner surface of the housing before they snap engage with the openings in the housing. It will be appreciated that the engagement between the stop element and the housing may be effected in other ways; for example, projections on the housing may snap engage with appropriate formations o n the stop element.

The hub and the stop element may be formed as plastics mouldings in such a way that the stop element is axially captive with the hub, the stop element and the hub being disengaged from each other during said final part of, or at the conclusion of, the delivery stroke of the plunger to allow the biasing element to drive the needle into the hollow plunger.

The manner in which the stop element and the hub interact with each other, the design features and production materials for these components and the biasing element may be as disclosed in International Patent Application No. PCT/GB02/01865 (WO-A-02/087669). Also the manner in which the plunger interacts with the stop element and the hub may be as disclosed in International Patent Application No. PCT/GB02/01865 (WO-A-02/087669). In particular, the plunger is, in some embodiments, associated, at its forward end, with a portion which is severable in response to movement of the plunger over the final part of, or at the conclusion of, its delivery stroke to allow retraction of the needle mounting hub into the hollow plunger.

The plunger may comprise a piston member and separate hollow rod arrangement as disclosed in our PCT Patent Application entitled “Prefilled Fluid Handling Device” having the same priority date, the entire disclosure of which is incorporated herein by this reference.

The stop element may be of generally cylindrical configuration and comprise a forward portion within the housing and a rearward portion of tapering configuration.

The invention will now be described by way of example only with reference to the accompanying drawings, in which:

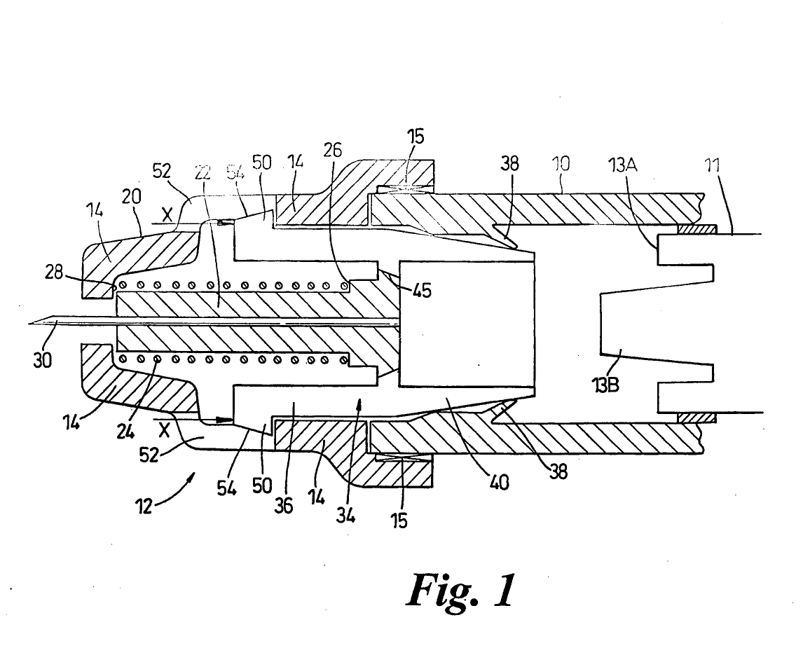

FIG. 1 is a schematic longitudinal sectional view of one form of needle unit and part of the barrel of a hypodermic syringe in accordance with the present invention;

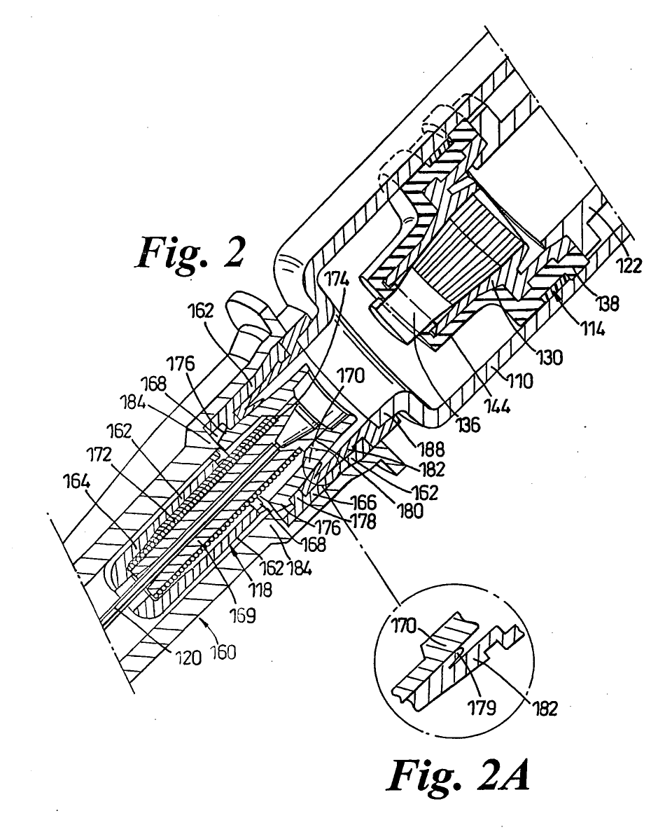

FIG. 2 is a perspective cut away view of another form of needle unit shown in conjunction with the barrel of a prefilled syringe.

The various aspects of the present invention may be implemented in a fluid handling device comprising a barrel for containing fluid to be delivered through a needle, a needle-mounting hub at one end of the barrel, a biasing element arranged to urge the hub inwardly of the barrel, a stop element blocking inward movement of the hub into the barrel, a hollow plunger which is movable within the barrel to deliver fluid from the barrel via the needle and is associated at its forward end with a portion which is severable in response to movement of the plunger over the final part of, or at the conclusion of, its delivery stroke to allow retraction of the needle-mounting hub into the hollow plunger, the hub and the stop element being formed as plastics mouldings in such a way that the stop element is axially captive with the hub, the hub and stop element being disengaged during said final part of, or at the conclusion of, the delivery stroke of the plunger to allow the biasing element to drive the needle into the hollow plunger.

Referring firstly to FIG. 1, a disposable medical syringe comprises a hollow barrel 10 which accommodates a plunger 11 generally of the form disclosed in International Patent Application No. PCT/GB02/01865 (WO-A-02/087669) or PCT/GB00/04573 (WP-A-0142104), the plunger comprising a rim portion 13A and a blocking portion 13B. The barrel 10 is provided at its forward end with a needle unit 12 comprising a housing 14 which is coupled to the barrel 10 by any suitable means 15 such as snap engaging formations or a screwthreaded connection which may, in each case, be irreversible so that the needle unit is permanently connected to the barrel.

A needle-mounting hub 22 is accommodated within the housing 14 and is encircled by a coiled compression spring 24 which engages at its forward end with a flange 28 of a conical section 20 of the housing and at its rear end with a radial shoulder 26 on the hub 22. The spring 24 serves to bias the hub 22 and needle 30 inwardly relative to the barrel 12 (and outwardly relative to the needle unit housing).

The hub 22 is of generally cylindrical configuration and has an axial bore for reception of the needle 30. The bore may be a throughbore and may be of tapering configuration to allow the needle to be fitted to the hub as a press-fit or by spin-welding.

Rearward movement of the hub and needle assembly (to the right as viewed in FIG. 1) is prevented by a stop element in the form of a crown 34 which is carried by hub 22. The crown 34 is of generally cylindrical configuration and comprises a forward portion 36 within the housing 14 and a rearward portion 40 of tapering, e.g. generally conical, configuration. The rearward portion 40 enters the forward end of the barrel 10 and co-operates with a lip seal 38 which flares radially inwardly and is resiliently flexible so that, on insertion of the crown portion 40, it is deflected radially outwardly so that it then bears resiliently against, and makes effective sealing engagement with, the outer peripheral surface of the crown portion 40. The lip seal 38 may be integral with the barrel which itself may be a plastics moulding.

The hub and crown are produced as plastics mouldings by two-shot moulding which serves to integrate the two components and render them captive with each other so that they effectively constitute a one-piece component designed to separate from each other at the interface between the hub and the crown when subjected to a predetermined forwardly directed axial force. At the interface 45 between the hub and crown, the components may have interfitting formations, e.g. mating frusto-conical surfaces, which may determine at least in part the force needed to separate the two components. Separation of the hub from the crown may be effected in the manner described in detail in International Patent Application No. PCT/GB02/101865.

The crown 34 is engaged with the housing 14 by snap engagement of laterally directed projections or latches 50 with openings 52 in the wall of the housing 14. The outer faces 54 of the projections 50 are inclined so that as the crown is inserted Into the housing, the projections may ride over the inner surface of the housing and the spring outwardly to effect snap engagement. This allows the crown, hub and spring to be assembled to the housing in a simple and effective manner by pushing the assembly of crown, hub and spring (against the biasing action of the spring) into the housing until the latches 50 spring into the openings 52 and prevent return movement of the assembly.

Once the crown, hub and spring assembly has been fitted to the housing, the needle unit is fitted to the barrel. This involves pushing the crown into the barrel against the resistance created by the lip seal 38. To avoid stressing the connection between the hub and the crown at this stage with the consequent risk of prematurely freeing the hub from the crown, the force needed to properly engage the crown with the lip seal is applied along a pathway which by-passes the hub/crown connection 45. More specifically, the force is applied directly to the crown (see arrows X) through the openings 52 by way of a sheath (not shown, but see FIG. 2 which illustrates the form that the sheath may take). The sheath serves to shield the needle and includes ribs arranged to contact the latches 50 via the openings 52.

After the needle unit has been assembled to the barrel and the syringe is about to be used to administer an injection, the sheath is removed.

Referring now to FIG. 2, the syringe shown is suitable for prefilled applications as disclosed in our PCT Patent Application entitled “Prefilled Fluid Handling Device” and having the same priority date, the entire disclosure of which is incorporated herein by this reference. The syringe includes a barrel 110, a piston unit 114 and a hollow rod 122 for displacing the piston member 114 towards the dispensing outlet 112 of the barrel so as to discharge a prefilled amount of drug or other component from the barrel and through the needle 120 of the needle unit 118. In FIG. 2, the needle unit is shown enclosed in a sheath 160 but, in practice, the sheath is removed before the contents of the barrel are discharged through the needle.

Features relating to the piston member 114, the barrel 110 and the rod 122 are described in said PCT Patent Application and reference should be made thereto for specific details. Briefly, the piston member comprises a central glass blocking portion 136 received in but severable or dislodgeable from a plastics rim portion 130 provided with a silicone rubber seal 138 having an integral extension 144 which overlies the forward face of the piston member to eliminate any risk of constituents of the plastics moulding contaminating the contents of the chamber 116.

The needle unit in FIG. 2 comprises a housing 162 having a relatively narrow forwardly extending section 164 and an enlarged trailing section 166, there being openings 163 in the housing 162 at the junction between the sections 164 and 166. As In the embodiment of FIG. 1, the housing 162 accommodates an assembly comprising a hub 169, a crown 170 and a coiled compression spring 172 which is trapped in a compressed, energy storing state between the forward end of the section 164 and a shoulder 174 on the hub 169. This assembly can be fitted to the housing in the same manner as described in connection with FIG. 1, i.e. by inserting the assembly until the latches snap enagage in the region of the openings 168. In this case, the snap engagement is between the latches 176 and an inwardly directed rib 178. The hub and crown are coupled together at the interface 180 in the manner previously described so that the hub can be released from the crown to allow needle retraction during the final part of or on completion of the forward stroke of the piston member 114.

In this embodiment, instead of providing the lip seal on the barrel (in this case, a glass barrel), the lip seal 179 (see the detail view, FIG. 2A) is provided on an intermediate plastics component 182 which couples the housing 162 of the needle unit to the dispensing outlet 112. The intermediate component 182 may engage with the outlet 112 in any suitable manner, e.g snap engagement or screwthreaded connection (irreversible or otherwise). The component 182 may be fitted to the barrel outlet 112 before it is engaged with the housing 162 or it may be initially fitted to the housing 162 and then coupled to the barrel outlet 112. To ensure that the interface 180 is not stressed while effecting sliding engagement with the lip seal 179, the sheath 160 can be used to apply the necessary force directly to the crown 170 via the openings 168. To this end, the sheath is provided with Internal ribs 184 which, when the sheath is fitted over the housing 162, contact the crown, e.g. the forward ends of the latches 176 at locations 186.

When the sheath 160 is in place enclosing the needle unit, the friction between the sheath 160 and the housing 162 holds the sheath 160 in position relative to the needle unit. Any forward force on the crown 170 is transmitted via the latches 176 to the internal ribs 184 of the sheath 160. Thus, provided that the frictional force between the sheath 160 and the housing 162 is not exceeded, the sheath 160 presents the forward movement of the crown 170 and any stress on the interface between the hub 169 and the crown 170. Accordingly, while the sheath 160 remains in place the possibility of the crown being inadvertently moved forward, separating from the hub 169 and causing premature retraction of the hub 169 and the needle 120 is reduced. This minimizes the possibility of the needle being inadvertently retracted, for example by the crown 170 being knocked as the needle unit is fitted to the barrel 110.

In practice, the prefilled syringe barrel will initially have a bung in its outlet 188 to retain the drug or other component within the chamber defined in the forward end of the barrel by the piston member 114. When the syringe is to be used to inject a patient, the bung is removed and the needle unit is fitted to the outlet 188 using the sheath to ensure that the interface between the hub and crown is not stressed during the fitting operation.

Whilst endeavouring in the foregoing specification to draw attention to those features of the invention believed to be of particular importance, it should be understood that the Applicant claims protection in respect of any patentable feature or combination of features disclosed herein and/or shown in the drawings whether or not particular emphasis has been placed on such feature or features.

1-18. (canceled)

19. A syringe comprising a barrel, an associated plunger and a needle unit, said needle unit comprising a housing connected to one end of the barrel, a needle-mounting hub, a biasing element arranged to urge the hub inwardly of the barrel, and a stop element blocking inward movement of the hub into the barrel until the hub is released from the stop element in response to the plunger reaching the final part of, or the conclusion of, its delivery stroke to allow retraction of the needle-mounting hub, characterized in that the barrel, or a part for connecting the needle housing to the barrel, is provided with a lip seal which contacts an outer peripheral surface of the stop element and which tends to deflect radially inwardly when a fluid within the barrel is pressurized during the delivery stroke of the plunger.

20. The syringe according to claim 1 wherein the lip seal is integral with the barrel or said connecting part.

21. The syringe according to claim 1 wherein the stop element is arranged to snap engage with the housing to couple the hub to the housing and to retain the biasing element in a stored energy condition.

22. The syringe according to claim 1 including a sheath for enclosing the needle of the needle unit, the housing including one or more openings through which the sheath and stop element can make contact.

23. The syringe according to claim 22 wherein the stop element is coupled to the needle mounting hub and the sheath and the stop element can make contact so that the sheath can be used to apply axial force to the stop element without significantly stressing the coupling between the stop element and the hub.

24. The syringe according to claim 22 wherein the sheath and the stop element can make contact such that the sheath restricts movement of the stop element, to prevent release of the needle mounting hub from the stop element.

25. The syringe according to claim 22 wherein one or more projections on the stop element engage wit the openings of the housing, the openings being arranged so that one or more portions of the sheath can abut with one or more projections.

26. The syringe according to claim 25 wherein the or each of the projections on the stop element engage with an inwardly directed rib or the like on the housing.

27. The syringe according to claim 25 wherein the or each of the projections on the stop element has an inclined outer face such that it may ride over the inner surface of the housing before snap engagement with the openings.

28. The syringe according to claim 1 wherein the plunger is hollow and retraction of the needle-mounting hub is into the hollow plunger.

29. The syringe according to claim 1 wherein once the housing or the connecting part is connected to the barrel, the force required to free the housing or the connecting part from the barrel is substantially in excess of the force required to effect the connection to the barrel.

30. The syringe according to claim 1 wherein the housing or the connecting part is engageable with the barrel with a snap fit.

31. The syringe according to claim 1 wherein the biasing element is a coiled compression spring, arranged in encircling relation with the needle.

32. The syringe according to claim 1 wherein the needle mounting hub and the stop element are formed as plastics molding such that the stop element is axially captive with the hub, the stop element and the hub being disengageable from each other during said final part of, at the conclusion of, the delivery stroke of the plunger to allow retraction of the needle-mounting hub.

33. The syringe according to claim 1, wherein the plunger comprises a piston member and a separate hollow rod.

34. The syringe according to claim 28 wherein the plunger is associated, at its forward end, with a portion which. is severable in response to movement of the plunger over the final part of, or at the conclusion of its delivery stroke, to allow retraction of the needle mounting hub into the hollow plunger.

35. The syringe according to claim 1 wherein the stop element is of generally cylindrical configuration and comprises a forward portion within the housing and a rearward portion of tapering configuration.
